(19) United States
(12) Plant Patent Application Publication
(10) Pub. No.: US 2004/0031078 P1
(43) Pub. Date: Feb. 12, 2004

(54) Polemonium reptans named 'Stairway to Heaven'

(76) Inventor: William Cullina, Hopkinton, MA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/213,866

(22) Filed: Aug. 7, 2002

(51) Int. Cl. A01H 5/00

(52) U.S. Cl. PLT/263

(57) ABSTRACT

A new and distinct cultivar of Polemonium plant named Stairway to Heaven, characterized by its distinct white and gray variegated foliage with violet flowers beginning in May and continuing to flower for 14-21 days in mid-spring.

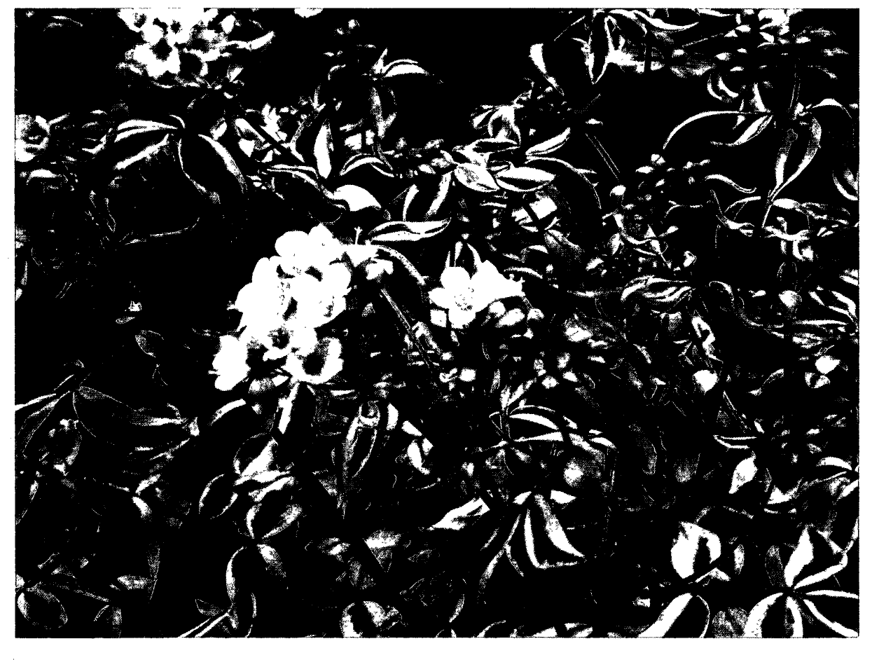

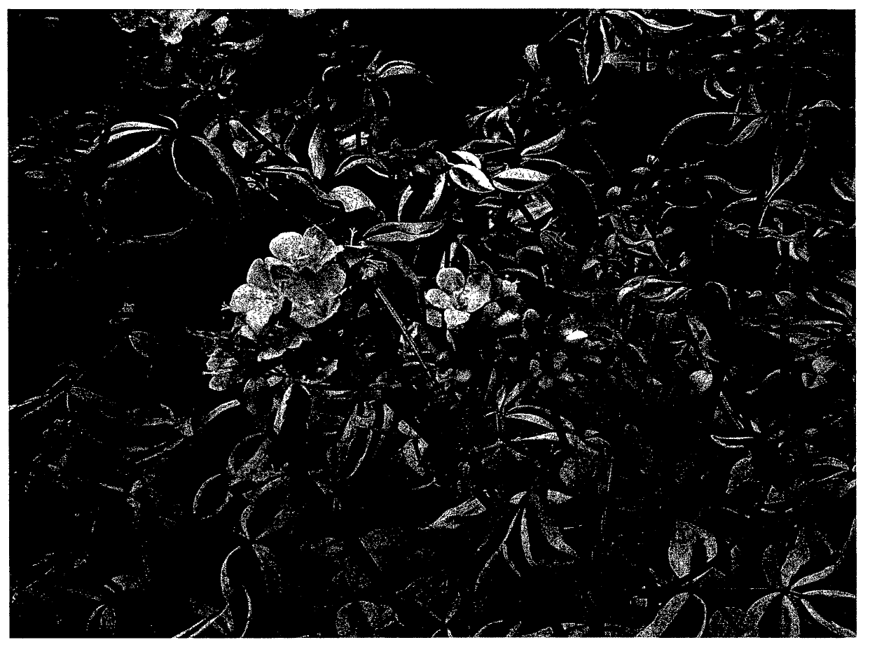

### BACKGROUND OF THE INVENTION

The present invention relates to a new distinctive cultivar of a plant of the hardy perennial herabaceous plant of the genus Polemonium, a member of the Polemoniaceae family, and the species Polemonium reptans, known by the cultivar name Stairway to Heaven. The species is commonly known and referred to by the commercial designation “Jacob's Ladder”.

### BRIEF SUMMARY OF THE INVENTION

The new variety was discovered at the New England Wild Flower Society's Native Plant Nursery in Framingham, Mass., as a naturally occurring sport or mutation of the species of Polemonium reptans and was discovered primarily due to its unique white and light green foliar variegation. During the winter, small, predominantly pink leaves cluster at soil level at the tips of active rhizomes.

Asexual reproduction of the new cultivar by tissue culture propagation and by division of the rhizome and roots taken at Just for Starters, Inc. in Eastford, Conn., has shown that the unique features of the new Polemonium are firmly fixed and retained through successive generations of asexual reproduction.

The new cultivar possesses many of the same characteristics as other cultivars of the species, including flower form and type, flowering habit, clump-forming habit and excellent vigor. Plants of the species, including the new cultivar, thrive best in moist, fertile soil and full sun to light shade.

The new cultivar Stairway to Heaven is particularly characterized by its variegated foliage which to the best knowledge of the inventors, is unique for the species. The main foliage color is light green but there is on each leaflet either a smooth or ragged marginal band of creamy white that extends ⅛^{th} to ½ of the way to the midvein. The center of a typical leaflet is medium green, and at the location where the white and green meet, there is an irregular band of pale gray-green produced from a blending of the others. The relative amount and distribution of the three colors (white, gray-green, and medium green) varies from leaflet to leaflet and from leaf to leaf. In general, the immature leaves have more marginal white banding, while on older and dehiscing leaves, the white changes to gray-green. Some leaflets are all white or white with a thin pocket of green or gray-green toward the center. The white areas of immature, expanding leaves taken on a light pink tone if grown in bright light, but the pink fades as the leaf becomes fully expanded. Flower color is identical with the parental species—violet blue. The habit of the flower is a clumping perennial with a mound of basal leaves and branching stems with smaller cauline leaves and flowers.

In addition to the described comparison with typical plants of the species, including the parent cultivar, Stairway to Heaven can be compared with the Polemonium reptans cultivar ‘Album’ which has white flowers and non-variegated foliage, and ‘Blue Pearl’ which is also non-variegated and has dark, blue-violet flowers. Additionally, Blue Pearl has a very different habit than Stairway to Heaven. The Blue Pearl is taller (24-28 inches), and the stem and leaves are stiffer.

The cultivar Stairway to Heaven has not been observed under all possible environmental conditions. The phenotype may vary somewhat with variations in environment such as temperature and light intensity. The following observations, measurements and comparisons describe plant grown in Framingham, Mass. under conditions which closely approximate those generally used in commercial practice.

The following traits have been repeatedly observed and are determined to be the distinctive and unique characteristics of Stairway to Heaven. These characteristics in combination distinguish Stairway to Heaven as a new and distinct cultivae.

### BRIEF DESCRIPTION OF THE DRAWING

The accompanying colored photographs illustrate the overall appearance of the new cultivar, showing the colors as true as it is reasonably possible to obtain in color reproductions of this type.

### DETAILED BOTANICAL DESCRIPTION

The following is a detailed description of Stairway to Heaven based on plants grown in Framingham, Mass. All color determinations and comparisons are based on The Royal Pantone Color Chart.

Classification:

Origin.—Naturally occurring seedling mutation discovered in the nursery of the New England Wild Flower Society, Framingham, Mass., USA.

Parentage.—Seedling from Polemonium reptans.

Plant:

Habit.—Clump forming, rhizomatous perennial.

Size.—Mature plants are 12-16 inches in height in bloom and 5-10 inches out of bloom with a spread of 14-18 inches.

Root System.—Moderately fleshy and fibrous roots originating from a creeping rhizome.

Plant vigor.—A perennial with excellent vigor. A flush of leaves and flowers appear as temperatures warm in spring, and with adequate moisture and fertility, new foliage will continue to be produced after flowering up until frost.

Blooming habit.—Plant sends up numerous branching stems from the rhizomes as weather warms in spring, each branchlet terminating in a cluster of from 1 to 15, half inch flowers.

Blooming period.—Total period of 14-21 days in mid-Spring. Depending on the weather, blooming can start as early as May 10^{th} or as late as June 5^{th}.

Method of asexual propagation.—Preferably by tissue culture propagation, although asexual reproduction through division of the rhizomes is possible.

Hardiness.—USDA hardiness zones 3-9.

Foliage:

Type.—Herbaceous.

Texture.—Medium.

Shape.—Generally rounded. Leaves are narrowly pinnate; leaflets are elliptical.

Length.—Leaves are 3 to 7 inches long; leaflets are 1 to 2 inches long.

Width.—Leaves are 2 to 3¾ inches wide; leaflets are ½ to ¾ inches wide.

Petiole length.—5 to 6 inches (basal), 0 to 1½ inches (cauline).

Margins.—Entire.

Apex (of leaflet).—Acute.

Base (of leaflet).—Rounded, sessile to rachis.

Flowers:

Form.—Panicle.

Borne.—In terminal and auxiliary clusters of 3-5 from the tops of flowering stems on 1 to 3 inch branchlets. Each main flowering stem can produce 20 to 30 flowers.

Petal count.—Five of equal size. Each is ¼ inches long.

Petal shape.—Rounded and lobed nearly to the middle, lobes entire.

Flower type.—Campanulate.

Flower size.—Petals are ¼ inch long; flowers are ½ inch in diameter, pedicel ¼ to ⅜^{th} inch long, slender.

Carpels.—One carpel, which is longer than the stamens and slightly longer than the corolla.

Stamens.—Shorter than corolla.

Color characteristics:

Foliage.—Immature leaves, especially those developing in temperatures under 70° F. have a temporary pink coloration (Pantone 236 C) that suffuses those areas that will later turn white. Mature leaves are irregularly variegated with a margin of white (no Pantone equivalent, but it is a creamy or eggshell white), a center of dark green (Pantone 5615C) and at the location where the white and dark green meet, there is a band of gray green (Pantone 5773 C). Older leaves eventually replace some of the white color with a pale yellow-green (Pantone 585 C).

Flowers.—The color of the flowers most closely approximates Pantone 270 C, though exact color varies with air temperature and sun exposure at time of anthesis.

It is claimed:

1. A new and distinct variety of Polemonium plant named Stairway to Heaven, substantially as illustrated and described.
